(19) United States
(12) Patent Application Publication
(10) Pub. No.: US 2004/0029194 A1
(43) Pub. Date: Feb. 12, 2004

(54) Method of identifying cancer markers and uses therefor in the diagnosis of cancer

(76) Inventors: Christopher Parish, Campbell (AU); Vivian Mae Cabalda-Crane, Evatt (AU)

Correspondence Address: Pillsbury Winthrop, Intellectual Property Group, 11682 El Camino Real, Suite 200, San Diego, CA, 92130, US

(21) Appl. No.: 10/333,348

(22) PCT Filed: Jul. 19, 2001
(86) PCT No.: PCT/AU01/00877

(30) Foreign Application Priority Data
Jul. 19, 2000 (AU) PQ 8861

(51) Int. Cl. G01N 33/574

(52) U.S. Cl. 435/7.23

(57) ABSTRACT

The present invention provides a mass spectrometry-based method of identifying a cancer marker in sera and uses of said cancer marker in diagnosing cancer in human and non-human subjects. The invention further provides isolated cancer markers.

### FIELD OF THE INVENTION

This invention relates to the diagnosis of cancer, and, more particularly, to a method for the diagnosis of cancer, using mass spectrometry (MS), in particular Matrix Assisted Laser Desorption/Ionisation Time of Flight Mass Spectrometry (hereinafter “MALDI-TOF MS”) or electrospray MS. The method of the present invention may be carried out to detect the presence of one or more cancerous cells or tumors in any human or animal subject, and optionally, to identify the type of cancer or malignant tumor, by assaying the blood or serum of said subject for an enhanced and/or reduced level of one or more molecular species, in particular a glycolipid, ganglioside, or oligosaccharide.

### GENERAL

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps, features, compositions and compounds.

The present invention is not to be limited in scope by the specific embodiments described herein, which are intended for the purposes of exemplification only. Functionally equivalent products, compositions and methods are clearly within the scope of the invention, as described herein.

The reference to any prior art document(s) in this specification is made merely for the purposes of further describing the instant invention and is not to be taken as an indication or admission that said document(s) forms part of the common general knowledge of a skilled person in Australia or elsewhere.

As used herein the words “from” or “of”, and the term “derived from” shall be taken to indicate that a specified product, in particular a molecule such as, for example, a polypeptide, protein, gene or nucleic acid molecule, antibody molecule, 1g fraction, or other molecule, or a biological sample comprising said molecule, may be obtained from a particular source, organism, tissue, organ or cell, albeit not necessarily directly from that source, organism, tissue, organ or cell.

As used herein, “cancer” shall be taken to mean any one or more of a wide range of benign or malignant tumors, including those that are capable of invasive growth and metastase through a human or animal body or a part thereof, such as, for example, via the lymphatic system and/or the blood stream. As used herein, the term “tumor” includes both benign and malignant tumors or solid growths, notwithstanding that the present invention is particularly directed to the diagnosis or detection of malignant tumors and solid cancers. Typical cancers include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans; and lymphoma (several), melanoma, sarcoma, and adenocarcinoma of animals.

In the context of the present invention as described herein and defined by the claims, the term “cancer marker” shall be taken to mean any molecule that is detectable in a blood fraction of a human or animal subject and is indicative of cancer in the subject, specifically a molecule that is produced by or is present on a cancer cell or a normal cell of the subject and whose level is modulated in the circulatory system of a subject having cancer compared to its level in the circulatory system of a healthy subject. The term “cancer marker” shall also be taken to include (i) a molecule that is expressed specifically by or on a cancer cell or whose expression is enhanced by or on a cancer cell compared to a normal cell; or (ii) a molecule that is expressed by or on a normal cell but not on a cancer cell, or is shed from a cancer cell, or whose expression is reduced by or on a cancer cells compared to a normal cell.

The term “cancer cell marker” will be understood by those skilled in the art to mean any molecule that is expressed specifically on a cancer cell or whose expression is enhanced on cancer cells compared to normal cells.

Typical cancer markers or cancer cell markers include, for example, protein, nucleic acid, lipid, glycolipid, glycoprotein, sugar (monosaccharide, disaccharide, oligosaccharide, etc), amongst others, the only requirement being that they are associated with a particular condition, phenotype, or cell type, and that they can be detected by an assay.

As used herein, a “glycolipid” is a lipid or fatty acid molecule having one or more carbohydrate moieties, including a ganglioside.

Those skilled in the art will be aware that a “ganglioside” is a glycosphingolipid that contains sialic acid (i.e. a glycolipid wherein a fatty acid-substituted sphingosine is linked to an oligosaccharide that comprises D-glucose, D-galactose, Nacetylgalactosamine and/or N-acetyineuraminic acid) and which is expressed in the majority of mammalian cell membranes. Gangliosides are mono-, di-, tri, or poly-sialogangliosides, depending upon the extent of glycosylation with sialic acid. In accordance with standard nomenclature, the terms “GMn”, “GDn”, “GTn”, are used, wherein “G” indicates a ganglioside; “M” indicates a monosialyl ganglioside, “D” indicates a disialyl ganglioside, and “T” indicates a trisialyl ganglioside; and wherein “n” is a numeric indicator having a value of at least 1, or an alphanumeric indicator having a value of at least 1a (e.g. 1a, 1b, 1c, etc), indicating the binding pattern observed for the molecule [Lehninger, In: Biochemistry, pp. 294-296 (Worth Publishers, 1981); Wiegandt, In: Glycolipids: New Comprehensive Biochemistry, pp. 199-260 (Neuberger et al., ed., Elsevier, 1985)].

The molecular masses of molecules referred to herein are in Daltons, and indicated by the abbreviation “Da”, consistent with accepted nomenclature.

### BACKGROUND OF THE INVENTION

In spite of numerous advances in medical research, cancer remains a major cause of death worldwide, and there is a need for rapid and simple methods for the early diagnoses of cancer, to facilitate appropriate remedial action by surgical resection, radiotherapy, chemotherapy, or other known treatment method. The availability of good diagnostic methods for cancer is also important to assess patient responses to treatment, or to assess recurrence due to re-growth at the original site or metastasis.

The characterization of cancer markers, such as, for example, oncogene products, growth factors and growth factor receptors, angiogenic factors, proteases, adhesion factors and tumor suppressor gene products, etc, can provide important information concerning the risk, presence, status or future behavior of cancer in a human or animal subject. Determining the presence or level of expression or activity of one or more cancer markers can assist the differential diagnosis of patients with uncertain clinical abnormalities, for example by distinguishing malignant from benign abnormalities. Furthermore, in patients presenting with established malignancy, cancer markers can be useful to predict the risk of future relapse, or the likelihood of response in a particular patient to a selected therapeutic course. Even more specific information can be obtained by analyzing highly specific cancer markers, or combinations of markers, which may predict responsiveness of a patient to specific drugs or treatment options.

Most known methods for the detection of cancer cells in a subject rely upon the detection of one or more high molecular weight or high molecular mass molecular species in a patient specimen. Immunological assays involve incubating the sample with an antibody molecule, particularly a monoclonal antibody, that binds specifically to a particular cancer cell marker in the sample. Alternatively, genetic tests involve the binding of a nucleic acid probe to nucleic acid in the sample that encodes a proteinaceous cancer cell marker, such as, for example, an oncoprotein. Before the advent of immunological or genetic assays, many cancer cell markers could only be detected or measured using conventional biochemical assay methods, which generally required large test samples and were therefore unsuitable for most clinical applications. In contrast, modern immunoassay and genetic assay techniques can detect and measure cancer cell markers in relatively much smaller samples, including biopsies, or serum.

Notwithstanding the advantages of existing assay techniques for identifying high molecular weight/mass cancer cell markers, such methods require the prior identification of the marker, the prior isolation of an appropriate probe to facilitate subsequent detection of the marker, and a time-consuming binding step in the assay procedure per se. A clear need exists for a rapid throughput method of detecting both high and low molecular weight/mass cancer markers (and cancer cell markers), and that facilitates sample handling and analysis (such as, for example, a process that does not require probe isolation or a binding reaction utilizing such a probe).

Additionally, immunoassays and genetic assays are generally used to determine the presence of a particular cancer cell marker in a sample, possibly because the antigen or nucleic acid detected is tumor-specific. A general method for detecting an enhanced or reduced level of any particular cancer marker is required.

Additionally, notwithstanding that a number of cancer-specific blood tests have been developed which depend upon the detection of tumor-specific antigens in circulation (Catalona et al., New England J. Med. 324, 1156-1161, 1991; Barrenetxea et al., Oncology 55, 447-449, 1998; Cairns et al., Biochim. Biophys. Acta 1423, C11-C18, 1999), efforts to utilize serum samples generally for cancer marker assays have met with limited success. Such limited success may be because a particular marker is not detectable in serum using immunoassay or genetic screening technology, or because changes in the level of a particular marker cannot be monitored in serum. Clearly, a need exists for a reproducible and reliable process for detecting the presence of cancer markers in serum samples.

### SUMMARY OF THE INVENTION

In work leading up to the present invention, the inventors sought to develop a general process for identifying both high and low molecular weight/mass cancer markers in the blood of human or animal subjects, and to develop related high throughput diagnostic methods for the detection of malignancies that were not limited in application to the detection of cancer cell markers or tumor-specific markers (i.e. cancer markers that are enhanced in tumor cells compared to normal cells), and/or did not depend upon the isolation of a molecular probe, such as, for example, an antibody or nucleic acid probe, and/or did not require a time-consuming binding step using such a molecular probe.

The inventors found that mass spectrometry (MS) was particularly suited to identifying a range of cancer markers in the blood or serum of a subject, specifically glycolipids or oligosaccharides whose abundance are modified (i.e. enhanced or decreased) in the circulatory system of a subject having cancer compared to a healthy subject.

Those skilled in the art will be aware that the adequacy of mass spectrometry, such as, for example, electrospray MS or MALDI-TOF MS, for analysis of any mass of compound, must be determined empirically. This is because the performance of a mass spectrometer is only partially defined by the mass resolution. Other important attributes are mass accuracy, sensitivity, signal-to-noise ratio, and dynamic range. The relative importance of the various factors defining overall performance depends on the type of sample and the purpose of the analysis, but generally several parameters must be specified and simultaneously optimized to obtain satisfactory performance for a particular application. The present inventors have now shown the utility of mass spectrometry to identifying cancer markers in blood or serum fractions, and to aiding the rapid and accurate diagnosis of cancer by an analysis of the modulation in cancer cell markers.

Accordingly, one aspect of the present invention provides a method of identifying a cancer marker comprising:

(i) separating a blood fraction from a human or animal subject having a cancer by mass spectrometry;

(ii) separating a blood fraction from a healthy human or animal subject by mass spectrometry; and

(iii) comparing the profile of molecular species at (i) and (ii) and identifying those molecular species having a modified level at (i) compared to (ii), wherein an enhanced or reduced level of said molecular species indicates that the molecular species is a cancer marker.

It will be apparent to the skilled person that the cancer marker identified in accordance with this aspect of the invention can be indicative of a specific type of cancer in a human or animal subject, and therefore aid the diagnosis or detection of that type of cancer. Accordingly, a second aspect of the invention provides a method for identifying a cancer marker that is indicative of a specific cancer, said method comprising:

(i) separating a blood fraction from a human or animal subject having a cancer by mass spectrometry;

(ii) separating a blood fraction from a human or animal subject having a cancer other than the cancer at (i) by mass spectrometry;

(iii) separating a blood fraction from a healthy human or animal subject by mass spectrometry; and

(iv) comparing the profile of molecular species at (i) and (ii) and (iii) and identifying those molecular species having a modified level at (i) or (ii) when compared to (iii), wherein said modified level indicates that the molecular species is a cancer marker that is indicative of a specific cancer.

In an alternative embodiment, this aspect of the invention provides a method for identifying a cancer marker that is indicative of a specific cancer, said method comprising:

(i) separating by mass spectrometry a panel of blood fractions from a human or animal subject wherein each member of said panel is from a subject having a distinct cancer;

(ii) separating a blood fraction from a healthy human or animal subject by mass spectrometry;

(iii) comparing the profiles of molecular species from each member of said panel of blood fractions at (i) to each other and to the profile of molecular species from the blood fraction at (ii); and

(iv) identifying from (iii) those molecular species having a modified level in one member of said panel at (i) when compared to the profile of the blood fraction at (ii), wherein said modified level indicates that the molecular species is a cancer marker that is indicative of a specific cancer.

A third aspect of the invention is directed to the diagnosis or detection of cancer in a human or animal subject, said method employing at least one mass spectrometric step.

In one embodiment of this aspect, the invention provides a method for diagnosing or detecting cancer in a human or animal subject comprising:

(i) separating a test sample comprising a blood fraction from a human or animal subject suspected of having a cancer by mass spectrometry;

(ii) separating a control sample comprising a blood fraction from a healthy subject by mass spectrometry; and

(iii) comparing the level of a cancer marker at (i) and (ii) wherein an enhanced or reduced level of said cancer marker in the test sample compared to the control sample indicates that the subject at (i) has a cancer.

It will be apparent from the description provided herein that, once a cancer marker has been identified using mass spectrometry in accordance with the invention, any art-recognized method can be employed to determine whether or not the cancer marker has a modified level in the subject, said modified level being diagnostic of cancer. Accordingly, mass spectrometry need not be employed in the actual diagnosis, provided that it has been employed in identifying the cancer marker. Accordingly, an alternative embodiment of the invention provides a method of diagnosing or detecting a cancer in a human or animal subject comprising:

(i) identifying a cancer marker by mass spectrometry in accordance with one or more embodiments described herein; and

(ii) determining the level of said cancer marker in a blood fraction from a human or animal subject suspected of having a cancer, wherein a modified level of said cancer marker compared to a healthy blood fraction indicates that the subject has cancer.

A fourth aspect of the invention provides an isolated cancer marker selected from the group consisting of:

(i) a glycolipid having a molecular mass in the range of 1439 to 1459 Da (average mass 1454 Da);

(ii) a glycolipid having a molecular mass in the range of 1587 to 1597 Da (average mass 1592 Da);

(iii) a glycolipid having a molecular mass in the range of 1616 to 1626 Da (average mass 1621 Da);

(iv) a glycolipid having a molecular mass in the range of 1671 to 1681 Da (average mass 1676 Da);

(v) a glycolipid having a molecular mass in the range of 1681 to 1691 Da (average mass 1686 Da);

(vi) a glycolipid or oligosaccharide having a molecular mass in the range of 809 to 819 Da (average mass 814 Da); and

(vii) a glycolipid or oligosaccharide having a molecular mass in the range of 1016 to 1026 Da (average mass 1021 Da).

### BRIEF DESCRIPTION OF THE DRAWINGS

FIG. 1 is a copy of a chromatogram of a MALDI-TOF mass spectrum obtained by separation of an ammonium sulfatelpyridine fraction of serum from BALB/c mice injected with dextran sulfate. A serum fraction that did not adsorb onto a C₁₈ Seppak cartridge was analyzed by MALDI-TOF MS, at a pulse voltage of 832 Volts. The x-axis indicates molecular mass (m/z), and the ordinate refers to the relative abundance of each molecular species as a percentage of the abundance of the most abundant species (i.e. m/z=839.9 Da±5 Da). Numbers at the top of each peak refer to the molecular mass, in Da, of that peak. The arrow indicates the position of a compound having a level that is enhanced by dextran sulfate treatment, and having a molecular mass (m/z) of about 1022 Da±5 Da.

FIG. 2 is a copy of a chromatogram of a MALDI-TOF mass spectrum obtained by separation of an ammonium sulfate/pyridine fraction of serum from normal BALB/c mice. A serum fraction that did not adsorb onto a C₁₈ Seppak cartridge was analyzed by MALDI-TOF MS, at a pulse voltage of 835 Volts. The x-axis indicates molecular mass (m/z), and the ordinate refers to the relative abundance of each molecular species as a percentage of the abundance of the most abundant species (i.e. m/z=840 Da±5 Da). Numbers at the top of each peak refer to the molecular mass, in Da, of that peak. The arrow indicates the position of the compound having a molecular mass (m/z) of about 1022 Da±5 Da (FIG. 1).

FIG. 3 is a copy of a chromatogram of a MALDI-TOF mass spectrum obtained by separation of an ammonium sulfate/pyridine fraction of serum from nude mice. A serum fraction that did not adsorb onto a C₁₈ Seppak cartridge was analyzed by MALDI-TOF MS, at a pulse voltage of 910 Volts. The x-axis indicates molecular mass (m/z), and the ordinate refers to the relative abundance of each molecular species as a percentage of the abundance of the most abundant species (i.e. m/z=839.8 Da±5 Da). Numbers at the top of each peak refer to the molecular mass, in Da, of that peak. The 1022 Da species (FIG. 1, FIG. 2) is not detectable in the serum fraction of nude mice under these conditions.

FIG. 4A is a copy of a chromatogram of a MALDI-TOF mass spectrum obtained by separation of an ammonium sulfate/pyridine fraction of serum from normal rats. A serum fraction that did not adsorb onto a C₁₈ Seppak cartridge was analyzed by MALDI-TOF MS, at a pulse voltage of 850 Volts. The x-axis indicates molecular mass (m/z), and the ordinate refers to the relative abundance of each molecular species as a percentage of the abundance of the most abundant species (i.e. m/z=865 Da±5 Da). Numbers at the top of each peak refer to the molecular mass, in Da, of that peak. The arrows indicate the positions of two compounds having levels that are reduced in subjects suffering from adenocarcinoma (FIG. 4B), and having molecular masses (m/z) of about 813.7 Da±5 Da and 1021.2 Da±5 Da. The latter-mentioned molecular mass corresponds to the 1022 Da species referred to in FIG. 1, FIG. 2, and FIG. 3.

FIG. 4B is a copy of a chromatogram of a MALDI-TOF mass spectrum obtained by separation of an ammonium sulfate/pyridine fraction of serum from rats carrying the highly metastatic rat mammary adenocarcinoma 13762 MAT. A serum fraction that did not adsorb onto a C₁₈ Seppak cartridge was analyzed by MALDI-TOF MS, at a pulse voltage of 850 Volts. The x-axis indicates molecular mass (m/z), and the ordinate refers to the relative abundance of each molecular species as a percentage of the abundance of the most abundant species. Numbers at the top of each peak refer to the molecular mass, in Da, of that peak. The two compounds indicated by arrows in FIG. 4A (m/z values of about 813.7 Da±5 Da and 1021.2 Da±5 Da) are not detectable.

FIG. 5A is a copy of a chromatogram of a MALDI-TOF mass spectrum obtained by separation of a chloroform/methanol extract of serum from normal rats. A serum fraction that elutes from a C₁₈ Seppak cartridge developed with chloroform/methanol was analyzed by MALDI-TOF MS, at a pulse voltage of 900 Volts. The x-axis indicates molecular mass (m/z), and the ordinate refers to the relative abundance of each molecular species as a percentage of the abundance of the most abundant species. Numbers at the top of each peak refer to the molecular mass, in Da, of that peak.

FIG. 5B is a copy of a chromatogram of a MALDI-TOF mass spectrum obtained by separation of a chloroform/methanol extract of serum from rats carrying the highly metastatic rat mammary adenocarcinoma 13762 MAT. A serum fraction that elutes from a C₁₈ Seppak cartridge developed with chloroform/methanol was analyzed by MALDI-TOF MS, at a pulse voltage of 900 Volts. The x-axis indicates molecular mass (m/z), and the ordinate refers to the relative abundance of each molecular species as a percentage of the abundance of the most abundant species (i.e. m/z=1621.1 Da±5 Da). Numbers at the top of each peak refer to the molecular mass, in Da, of that peak. The four compounds indicated by arrows (m/z values of about 1454 Da±5 Da, 1592 Da±5 Da, 1621 Da±5 Da, and 1687 Da±5 Da) are not detectable at enhanced levels in the sera of normal rats.

FIG. 6A is a copy of a chromatogram of a MALDI-TOF mass spectrum obtained by separation of a chloroform/methanol extract of serum from normal rats. A serum fraction that elutes from a C₁₈ Seppak cartridge developed with chloroform/methanol was analyzed by MALDI-TOF MS, at a pulse voltage of 900 Volts. The x-axis indicates molecular mass (m/z), and the ordinate refers to the relative abundance of each molecular species as a percentage of the abundance of the most abundant species. Numbers at the top of each peak refer to the molecular mass, in Da, of that peak. Two compounds are indicated by arrows (m/z values of about 1185 Da±5 Da, and 1676 Da±5 Da).

FIG. 6B is a copy of a chromatogram of a MALDI-TOF mass spectrum obtained by separation of a chlorofom/methanol extract of serum from rats carrying the highly metastatic rat mammary adenocarcinoma 13762 MAT. A serum fraction that elutes from a C₁₈ Seppak cartridge developed with chloroform/methanol was analyzed by MALDI-TOF MS, at a pulse voltage of 900 Volts. The x-axis indicates molecular mass (m/z), and the ordinate refers to the relative abundance of each molecular species as a percentage of the abundance of the most abundant species (i.e. m/z=1676 Da±5 Da). Numbers at the top of each peak refer to the molecular mass, in Da, of that peak. Two compounds are indicated by arrows (m/z values of about 1185 Da±5Da, and 1676 Da±5 Da), and the level of the 1676 Dalton species is elevated in the sera of rats having cancer compared to the level in sera of normal rats, when standardized against the level of the 1185 Dalton species.

### DETAILED DESCRIPTION OF THE PREFERRED EMBODIMENTS

One aspect of the present invention provides a method of identifying a cancer marker comprising:

(i) separating a blood fraction from a human or animal subject having a cancer by mass spectrometry;

(ii) separating a blood fraction from a healthy human or animal subject by mass spectrometry; and

(iii) comparing the profile of molecular species at (i) and (ii) and identifying those molecular species having a modified level at (i) compared to (ii), wherein an enhanced or reduced level of said molecular species indicates that the molecular species is a cancer marker.

Preferably, the present invention is directed to the identification of cancer markers in respect of a cancer selected from the group consisting of carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans; and lymphoma (several), melanoma, sarcoma, and adenocarcinoma of animals. In a particularly preferred embodiment of the invention, the cancer is a carcinoma, more preferably an adenocarcinoma.

Those skilled in the art will be aware that mass spectrometry is an analytical technique for the accurate determination of molecular weights, the identification of chemical structures, the determination of the composition of mixtures, and qualitative elemental analysis. In operation, a mass spectrometer generates ions of sample molecules under investigation, separates the ions according to their mass-to-charge ratio, and measures the relative abundance of each ion. Preferably, the mass spectrometry system used in performing the present invention is MALDI-TOF MS or electrospray MS.

The general steps in performing a mass-spectrometric analysis are as follows:

(i) create gas-phase ions from a sample;

(ii) separate the ions in space or time based on their mass-to-charge ratio; and

(iii) measure the quantity of ions of each selected mass-to-charge ratio.

The term “separating a blood fraction by mass spectrometry”, or similar term used herein, shall be taken to include any one or more of said steps.

Time-of-flight (TOF) mass spectrometers, such as, for example, those described in U.S. Pat. No. 5,045,694 and U.S. Pat. No. 5,160,840, generate ions of sample material under investigation and separate those ions according to their mass-to-charge ratio by measuring the time it takes generated ions to travel to a detector. TOF mass spectrometers are advantageous because they are relatively simple, expensive instruments with virtually unlimited mass-to-charge ratio range. TOF mass spectrometers have potentially higher sensitivity than scanning instruments because they can record all the ions generated from each ionization event. TOF mass spectrometers are particularly useful for measuring the mass-to-charge ratio of large organic molecules where conventional magnetic field mass spectrometers lack sensitivity.

The flight time of an ion accelerated by a given electric potential is proportional to its mass-to-charge ratio. Thus the time-of-flight of an ion is a function of its mass-to-charge ratio, and is approximately proportional to the square root of the mass-to-charge ratio. Assuming the presence of only singly charged ions, the lightest group of ions reaches the detector first and are followed by groups of successively heavier mass groups.

TOF mass spectrometers thus provide an extremely accurate estimate of the molecular mass of a molecular species under investigation, and the error, generally no more than ±5 Da, is largely a consequence of ions of equal mass and charge not arriving at the detector at exactly the same time. This error occurs primarily because of the initial temporal, spatial, and kinetic energy distributions of generated ions that lead to broadening of the mass spectral peaks, thereby limiting the resolving power of TOF spectrometers. The initial temporal distribution results from the uncertainty in the time of ion formation.

The certainty of time of ion formation is enhanced by utilizing pulsed ionization techniques, such as, for example, plasma desorption and laser desorption, that generate ions during a very short period of time and result in the smallest initial spatial distributions, because ions originate from well defined areas on the sample surface and the initial spatial uncertainty of ion formation is negligible.

Pulsed ionization such as plasma desorption (PD) ionization and laser desorption (LD) ionization generate ions with minimal uncertainty in space and time, but relatively broad initial energy distributions. Because long pulse lengths can seriously limit mass resolution, conventional LO typically employs sufficiently short pulses (frequently less than 10 nanoseconds) to minimize temporal uncertainty.

The performance of LD is enhanced by the addition of a small organic matrix molecule to the sample material, that is highly absorbing, at the wavelength of the laser (i.e. Matrix-assisted laser desorption/ionization, hereinafter “MALDI”). The matrix facilitates desorption and ionization of the sample. MALDI is particularly advantageous in biological applications since it facilitates desorption and ionization of large biomolecules in excess of 100,000 Da molecular mass without their fractionation. A preferred matrix for performing the instant invention comprises 2-(4-hydroxyphenylazo)benzoic acid (HABA), also known as 4-hydroxybenzene-2-carboxylic acid.

In MALDI, samples are usually deposited on a smooth metal surface and desorbed into the gas phase as the result of a pulsed laser beam impinging on the surface of the sample. Thus, ions are produced in a short time interval, corresponding approximately to the duration of the laser pulse, and in a very small spatial region corresponding to that portion of the solid matrix and sample which absorbs sufficient energy from the laser to be vaporized. MALDI provides a near-ideal source of ions for time-of-flight (TOF) mass spectrometry, particularly where the initial ion velocities are small. Considerable improvements in mass resolution are obtained using pulsed ion extraction in a MALDI ion source. Ion reflectors (also called ion mirrors and reflectrons, consisting of one or more homogeneous, retarding, electrostatic fields) are also known to compensate for the effects of the initial kinetic energy distribution of the analyte ions, particularly when positioned at the end of the free-flight region. Additional improvements to MALDI are known in the art with respect to the production of ions from surfaces, by improving resolution, increasing mass accuracy, increasing signal intensity, and reducing background noise, such as, for example, those improvements described in U.S. Pat. No. 6,057,543.

The present invention encompasses the use of all modified MALDI-TOF MS systems to determine a cancer marker in a blood fraction and/or to aid the diagnosis of cancer.

Electrospray MS, or electrospray ionization MS, is used to produce gas-phase ions from a liquid sample matrix, to permit introduction of the sample into a mass spectrometer. Electrospray MS is therefor useful for providing an interface between a liquid chromatograph and a mass spectrometer. In electrospray MS, a liquid analyte is pumped through a capillary tube (hereinafter “needle”), and a potential difference (e.g. three to four thousand Volts) is established between the tip of the needle and an opposing wall, capillary entrance, or similar structure. The stream of liquid issuing from the needle tip is diffused into highly-charged droplets by the electric field, forming the electrospray. An inert drying gas, such as, for example, dry nitrogen gas, may also be introduced through a surrounding capillary to enhance nebulization of the fluid stream. The electrospray droplets are transported in an electric field and injected into the mass spectrometer, which is maintained at a high vacuum. Through the combined effects of a drying gas and vacuum, the carrier liquid in the droplets evaporates gradually, giving rise to smaller, increasingly unstable droplets from which surface ions are liberated into the vacuum for analysis. The desolvated ions pass through sample cone and skimmer lenses, and after focusing by a RF lens, into the high vacuum region of the mass-spectrometer, where they are separated according to mass and detected by an appropriate detector (e.g., a photo-multiplier tube). Preferred liquid flow rates of 20-30 microliters/min are used, depending on the solvent composition. Higher liquid flow rates may result in unstable and inefficient ionization of the dissolved sample, in which case a pneumatically-assisted electrospray needle may be used.

Those skilled in the art will also be aware that it is necessary to prepare the blood fraction undergoing analysis, for introduction into the MS environment. Preferably, the sample is at least desalted essentially as described in Example 1. More preferably, the sample is fractionated prior to analysis using at least one standard chromatographic separation or purification step. In cases where MALDI-TOF MS is employed, the sample will be mixed with a suitable matrix and dried, whereas in the case of electrospray MS, the sample will be injected directly as a liquid sample in an appropriate carrier solution.

The term “subject having a cancer” will be understood to mean that the subject has exhibited one or more symptoms associated with a cancer, or has previously been diagnosed as having cancer at the time of obtaining the blood fraction used as a test sample in the inventive method.

As used herein, the term “healthy subject” shall be taken to mean a subject that has not exhibited any symptoms associated with cancer when the blood fraction was taken, or is in remission from the symptoms associated with cancer when the blood fraction was taken, or has not exhibited any metastases of a previously-diagnosed tumor in the blood or serum at the time when the blood fraction was taken. Accordingly, the “healthy subject” need not be distinct from the subject suspected of having cancer. For example, a particular individual, such as, for example an individual at risk of developing cancer, may provide blood fractions at different times, in which case an early blood fraction taken prior to any symptom development may be used as a control sample against a later blood fraction being tested. Alternatively, a blood fraction taken from a subject in remission, or following treatment, may be used as a control sample against a blood fraction from the same subject taken earlier or later, such as, for example, to monitor the progress of the disease.

By “control sample” is meant a sample having a known composition or content of a particular integer against which a comparison to a test sample is made. The only requirement for the source of a control sample is that it does not contain a level of the cancer marker being detected consistent with disease.

It will be apparent from the preceding description that it is not usual to subject whole blood or whole cells to fractionation by mass spectrometry. Instead, fractions containing the molecular species to be analyzed, in particular, blood fractions comprising glycolipid or oligosaccharide, and more preferably, blood fractions comprising ganglioside, are loaded into the mass spectrometer. Such blood fractions can be prepared by standard methods known to those skilled in the art or prepared according to the methods described herein without undue experimentation.

A “blood fraction” means any derivative of blood, and shall be taken to include a supernatant or precipitate of blood, a serum fraction or plasma fraction, a buffy coat fraction, a fraction enriched for T-cells, a fraction enriched for platelets, a fraction enriched for platelets erythrocytes, a fraction enriched for basophils, a fraction enriched for eosinophils, a fraction enriched for lymphocytes, a fraction enriched for monocytes, a fraction enriched for neutrophils, or any partially-purified or purified component or blood whether or not in admixture with any other component of blood. Blood fractions may be obtained, for example, by treatment of blood with a precipitant (e.g. low temperature, acid, base, ammonium sulfate, polyethylene glycol, etc), or fractionation by chromatography (e.g. size exclusion, ion exchange, hydrophobic interaction, reverse phase, mass spectrometry, etc).

Particularly preferred blood fractions for use in performing the invention are serum fractions. In the present context, the term “serum fraction” means a sample derived from serum. Exemplary serum fractions include a plasma protein fraction (e.g. albumin fraction, fibrinogen (factor I) fraction, serum globulin fraction, factor V fraction, factor VIII fraction, or prothrombin complex fraction comprising factors VII, IX and X), a cryosupernatant or cryoprecipitate of plasma, a cryosupernatant or cryoprecipitate of fresh frozen plasma, a cryosupernatant or cryoprecipitate of a plasma fraction, or any partially-purified or purified component of serum whether or not in admixture with any other serum component. Serum fractions may be obtained, for example, by treatment of serum with a precipitant (e.g. low temperature, acid, base, ammonium sulfate, polyethylene glycol, etc), or by fractionation using chromatography (e.g. size exclusion, ion exchange, hydrophobic interaction, reverse phase, mass spectrometry, etc).

Preferably, this aspect of the invention further includes the first step of obtaining the blood fraction, preferably as a precipitate of serum, and/or preferably desalted, and/or preferably fractionated by hydrophobic interaction chromatography, such as, for example using a polycarbon matrix. Other means of obtaining the blood fraction in accordance with procedures known to the skilled person are clearly contemplated herein.

Because the method of the present invention is performed on blood or serum, it is convenient to perform and non-invasive.

The “molecular species”, identified in accordance with the present invention is a glycolipid, in particular a ganglioside or oligosaccharide compound. Preferably, but not necessarily, the molecular species is immune system dependent in so far as it requires the presence of an activated or functional immune system for its expression. As exemplified herein, a cancer marker of 1021 Da (molecular mass range of 1016-1026 Da) is detected in the serum fraction of mice treated with dextran sulfate but not in the serum fraction of nude mice, suggesting that expression of that marker is strongly immune system dependent and that tumorigenesis reduces its expression or causes its shedding from the cell surface. As used herein, the term “immune system dependent” includes a requirement for T-cell function to ensure that the cancer marker is expressed, or an indication that a particular cancer marker is normally expressed on a T-cell, or alternatively, shed from a T-cell at any stage during tumorigenesis, preferably prior to metastases.

By “comparing the profile of molecular species” is meant that the molecular mass profile of the blood fraction from the cancer sample is compared or aligned to the molecular mass profile of the blood fraction from the cancer sample and the differences noted. Those skilled in the art will be aware that conditions for mass spectrometry of a sample can be manipulated to ensure that the peak height of a particular molecular species, or the area of a particular peak, is proportional to the abundance of that molecular species in the sample. Accordingly, it is not strictly necessary to conduct a further assay of a collected peak sample to determine the abundance of the molecular species therein, because the molecular mass spectra of two samples may be overlaid to determine the differences in peak heights. Notwithstanding that this may be the case, the present invention clearly includes the step of determining the abundance of any candidate molecular species identified in either the blood fraction from the subject having cancer or the blood fraction from the healthy subject, and/or the relative abundance of a molecular species in said blood fractions. This includes determining the abundance or relative abundance of that molecular species in the blood or serum from which the blood fraction is derived. Standard assays for determining the level of ganglioside or oligosaccharide in a sample may be employed, such as, for example, an immunochemical analysis of the peak fraction.

Preferably, the method according to this aspect of the invention includes the further characterization of the cancer marker, in particular according to its molecular mass. The molecular mass (Da) of the cancer marker is readily determined by mass spectrometry against standard compounds of known molecular mass, with a maximum error in the estimated molecular mass of ±5 Da, more preferably ±4 Da, even more preferably ±3 Da, still more preferably ±2 Da, and even still more preferably ±1 Dalton.

The cancer marker may also be further characterized structurally, such as, for example, by fragmentation studies using ESI/MS/MS/QTOF, or enzymatic digestion of glycosyl or lipid moieties, amongst other techniques known to those skilled in the art.

The present inventors have identified a number of cancer markers whose abundance is enhanced in the serum of a subject having cancer, as follows:

(i) a glycolipid having a molecular mass in the range of 1439 to 1459 Da (average mass 1454 Da);

(ii) a glycolipid having a molecular mass in the range of 1587 to 1597 Da (average mass 1592 Da);

(iii) a glycolipid having a molecular mass in the range of 1616 to 1626 Da (average mass 1621 Da);

(iv) a glycolipid having a molecular mass in the range of 1671 to 1681 Da (average mass 1676 Da); and

(v) a glycolipid having a molecular mass in the range of 1681 to 1691 Da (average mass 1686 Da).

Of these five cancer markers, species (iv) is also detectable, albeit at a low level, in the serum fraction of a healthy subject, whilst the remaining markers are not detectable using standard MALDI-TOF MS.

The present inventors have also identified two cancer markers whose abundance is reduced in the serum of a subject having cancer, as follows:

(i) a glycolipid or oligosaccharide having a molecular mass in the range of 809 to 819 Da (average mass 814 Da); and

(ii) a glycolipid or oligosaccharide having a molecular mass in the range of 1016 to 1026 Da (average mass 1021 Da).

It will be apparent to the skilled person that the cancer marker identified in accordance with this aspect of the invention can be indicative of a specific type of cancer in a human or animal subject, and therefore aid the diagnosis or detection of that type of cancer. For example, in vitro studies indicate that different cancer types secrete a unique spectrum of gangliosides (Kong et al., Biochim. Biophys. Acta 1394, 43-56, 1998), indicating that the mass spectrometry approach described herein may also be used to assist in the diagnosis of the type of cancer present in a patient.

The only requirement to identifying a cancer-specific cancer marker using mass spectrometry is to screen a sufficient number of blood fractions to determine that the particular marker is restricted to a particular type of cancer cell, and not generic to all cancer cell types. At least two blood fractions from subjects having distinct cancers are required to facilitate this determination. Preferably, several blood fractions are employed.

Accordingly, a second aspect of the invention provides a method for identifying a cancer marker that is indicative of a specific cancer, said method comprising:

(i) separating a blood fraction from a human or animal subject having a cancer by mass spectrometry;

(ii) separating a blood fraction from a human or animal subject having a cancer other than the cancer at (i) by mass spectrometry;

(iii) separating a blood fraction from a healthy human or animal subject by mass spectrometry;

(iv) comparing the profile of molecular species at (i) and (ii) and (iii); and

(v) identifying those molecular species having a modified level at (i) or (ii) when compared to (iii), wherein said modified level indicates that the molecular species is a cancer marker that is indicative of a specific cancer.

In accordance with this embodiment, there is no requirement for the level of the molecular species to vary in all three blood fractions (i.e. the two samples from the subjects having cancers, and the single control blood fraction from the healthy subject). This is because a cancer marker that is indicative of a specific cancer will, by definition, vary in amount only for a single cancer, and be present at a normal level in other samples. In contrast, those molecular species that are not present at a different level in the two cancer-derived samples will not be indicative of a specific cancer, notwithstanding that, if they differ in amount to their levels in normal cells, they will be cancer markers falling within the scope of the invention described herein. Those molecular species that are not present at a different level in any of the samples analyzed will not be cancer markers.

In an alternative embodiment, this aspect of the invention provides a method for identifying a cancer marker that is indicative of a specific cancer, said method comprising:

(i) separating by mass spectrometry a panel of blood fractions from a human or animal subject wherein each member of said panel is from a subject having a distinct cancer;

(ii) separating a blood fraction from a healthy human or animal subject by mass spectrometry;

(iii) comparing the profiles of molecular species from each member of said panel of blood fractions at (i) to each other and to the profile of molecular species from the blood fraction at (ii); and

(iv) identifying from (iii) those molecular species having a modified level in one member of said panel at (i) when compared to the profile of the blood fraction at (ii), wherein said modified level indicates that the molecular species is a cancer marker that is indicative of a specific cancer.

As used herein, the term “distinct cancer” shall be taken to mean a different cancer type.

The modified level of any particular molecular species, in particular, the modified level of a cancer marker, on a tumor cell compared to a normal cell can also be diagnostic of cancer. Accordingly, a third aspect of the invention relates to the diagnosis or detection of cancer in a human or animal subject.

In one embodiment of this aspect, the invention provides a method for diagnosing or detecting cancer in a human or animal subject comprising:

(i) separating a test sample comprising a blood fraction from a human or animal subject suspected of having a cancer by mass spectrometry;

(ii) separating a control sample comprising a blood fraction from a healthy subject by mass spectrometry; and

(iii) comparing the level of a cancer marker at (i) and (ii) wherein an enhanced or reduced level of said cancer marker in the test sample compared to the control sample indicates that the subject at (i) has a cancer.

In the present context, term “subject suspected of having a cancer” shall be taken to indicate merely that the subject is being tested, and is not to be taken as indicating in any manner that the invention requires the subject to exhibit any symptoms associated with a particular cancer, tumorigenesis or metastases, or that any prior diagnostic test must be employed prior to the inventive diagnostic test described herein. In fact, as the present invention is particularly amenable to the early diagnosis of cancer, no prior testing or evaluation is essential to performing the invention, notwithstanding that such additional testing may be employed in the interests of confirming any diagnosis.

The cancer is preferably selected from the group consisting of carcinoma, lymphoma, sarcoma, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma, lymphoma (several), melanoma, sarcoma, and adenocarcinoma. In a particularly preferred embodiment of the invention, the cancer is a carcinoma, more preferably an adenocarcinoma.

It will be apparent from the preceding discussion that the diagnostic method described herein is not limited to the diagnosis of cancer, however can be applied to monitoring the progress of the disease in a particular subject, by comparing the level of one or more cancer markers in the subject over time. In the case of a patient in remission, a sample taken early in remission can be used as a standard for comparison against later blood fractions, to determine the status of the subject, since any further modification to the level of a cancer marker may indicate that the period of remission has ended. Similarly, for a patient who has undergone treatment successfully leading to a remission or cure, or who has not exhibited any metastases, a sample taken shortly after treatment or prior to metastases can be used as a standard for comparison against later blood fractions, to determine whether or not the subject has suffered recurrence or metastases of the tumor, since any modified level of a cancer marker may indicate recurrence or metastases.

Sample preparation for mass spectrometry in performing the instant cancer diagnostic method are essentially the same as described supra for the identification of cancer markers.

Comparison of the cancer marker at sub-paragraph (iii) of the method recited supra can also be performed as described in the preceding discussion, with reference to the identification of cancer markers using mass spectrometry.

Preferably, this aspect of the invention further includes the first step of obtaining the blood fraction, preferably as a precipitate of serum, and/or preferably desalted, and/or preferably fractionated by hydrophobic interaction chromatography, such as, for example using a polycarbon matrix. Other means of obtaining the blood fraction in accordance with procedures known to the skilled person are clearly contemplated herein.

Preferably, the method according to this aspect of the invention includes the further characterization of the cancer marker, in particular according to its molecular mass. The molecular mass (Da) of the cancer marker is readily determined by mass spectrometry against standard compounds of known molecular mass, with a maximum error in the estimated molecular mass of ±5 Da, more preferably ±4 Da, even more preferably ±3 Da, still more preferably ±2 Da, and even still more preferably ±1 Dalton.

Preferably, the cancer marker that is compared in accordance with the diagnostic method of the invention is selected from the group consisting of:

(i) a glycolipid having a molecular mass in the range of 1439 to 1459 Da (average mass 1454 Da);

(ii) a glycolipid having a molecular mass in the range of 1587 to 1597 Da (average mass 1592 Da);

(iii) a glycolipid having a molecular mass in the range of 1616 to 1626 Da (average mass 1621 Da);

(iv) a glycolipid having a molecular mass in the range of 1671 to 1681 Da (average mass 1676 Da);

(v) a glycolipid having a molecular mass in the range of 1681 to 1691 Da (average mass 1686 Da);

(vi) a glycolipid or oligosaccharide having a molecular mass in the range of 809 to 819 Da (average mass 814 Da); and

(vii) a glycolipid or oligosaccharide having a molecular mass in the range of 1016 to 1026 Da (average mass 1021 Da).

It will be apparent from the description provided herein that, once a cancer marker has been identified using mass spectrometry in accordance with the invention, any art-recognized method can be employed to determine whether or not the cancer marker has a modified level in the subject, said modified level being diagnostic of cancer. Accordingly, mass spectrometry need not be employed in the actual diagnosis, provided that it has been employed in identifying the cancer marker.

Accordingly, an alternative embodiment of the invention provides a method of diagnosing or detecting a cancer in a human or animal subject comprising:

(i) identifying a cancer marker by mass spectrometry in accordance with one or more embodiments described herein; and

(ii) determining the level of said cancer marker in a blood fraction from a human or animal subject suspected of having a cancer, wherein a modified level of said cancer marker compared to a healthy blood fraction indicates that the subject has cancer.

Once identified and characterized, standard methods may be employed to determine the level of the cancer marker in a blood fraction, including mass spectrometry, high pressure liquid chromatography (HPLC)-mass spectrometry, hydrophobic interaction chromatography, size exclusion chromatography, ion exchange chromatography, or other art-recognized method.

For example, monoclonal antibodies can be prepared against a peak fraction from mass spectrometry comprising the cancer marker, in particular a ganglioside, and then used in standard immunoassay techniques for the subsequent diagnosis of cancer.

In performing this embodiment of the invention, mice or other mammals can be pre-treated by injection with low doses of cyclophosphamide (15 mg/Kg animal body weight) to reduce their suppressor cell activity, and then immunized with various doses of liposome preparations containing gangliosides, at short intervals (i.e. between 3-4 days and one week), essentially as described in U.S. Pat. No. 5,817,513. Immunizations can be performed by subcutaneous, intravenous, or intraperitoneal injection, in accordance with standard procedures. Before and during the immunization period, animal blood serum samples are taken for monitoring antibody titers generated in the animals against the gangliosides used as antigens by any known immunoassay method detecting an antigen-antibody reaction. In general, about 5-9 accumulative doses of a liposome preparation at short time intervals will facilitate an antibody response to the ganglioside. Mice with serum antibody titers against gangliosides receive a new immunization with the liposome preparations, about three days before obtaining antibody producing cells, and then the antibody producing cells, preferably spleen cells, are isolated. These cells are fused with myeloma cells to produce hybridomas in accordance with standard procedures for preparing monoclonal antibodies. The titres of the monoclonal antibodies produced by the hybridomas are then tested by immunoassay methods. Preferably, an immuno-enzymatic assay is employed, in which hybridoma supernatants bind to a test sample containing the ganglioside antigen and then antigen-antibody binding is detected using a second enzyme labelled antibody that binds to the monoclonal antibody. Once the desired hybridoma is selected and sub-cloned, such as, for example, by limiting dilution, the resulting monoclonal antibody can be amplified in vitro in an adequate medium, during an appropriate period, followed by the recovery of the desired antibody from the supernatant. The selected medium and the adequate culture time period are known to the skilled person, or easily determined.

Another production method comprises the injection of the hybridoma into an animal, for example, syngeneic mice. Under these conditions, the hybridoma causes the formation of non-solid tumors, which will produce a high concentration of the desired antibody in the blood stream and the peritoneal exudate (ascites) of the host animal.

Standard immunoassays are then used to assay for the presence of the ganglioside antigen in a blood fraction obtained from a subject suspected of having cancer. By comparison of the test result to a blood fraction obtained from a healthy subject, an appropriate diagnosis can be made.

A fourth aspect of the invention provides an isolated cancer marker selected from the group consisting of:

(i) a glycolipid having a molecular mass in the range of 1439 to 1459 Da (average mass 1454 Da);

(ii) a glycolipid having a molecular mass in the range of 1587 to 1597 Da (average mass 1592 Da);

(iii) a glycolipid having a molecular mass in the range of 1616 to 1626 Da (average mass 1621 Da);

(iv) a glycolipid having a molecular mass in the range of 1671 to 1681 Da (average mass 1676 Da);

(v) a glycolipid having a molecular mass in the range of 1681 to 1691 Da (average mass 1686 Da);

(vi) a glycolipid or oligosaccharide having a molecular mass in the range of 809 to 819 Da (average mass 814 Da); and

(vii) a glycolipid or oligosaccharide having a molecular mass in the range of 1016 to 1026 Da (average mass 1021 Da).

By “isolated” means substantially free of conspecific glycolipids or oligosaccharides, such as, for example, determined by mass spectrometry under the conditions defined herein. By virtue of the high resolution of MALDI-TOF MS, it will be understood by the skilled person that the glycolipid or oligosaccharide peaks exemplified herein correspond to isolated molecular species as defined herein.

Preferably, the isolated cancer marker is an immune system dependent ganglioside or oligosaccharide, and more preferably, a T-cell dependent ganglioside or oligosaccharide.

The examples presented below demonstrate that MALDI-TOF mass spectrometry can be used to detect immune system-derived molecular species, the loss of some molecular species in cancer-bearing animals and the appearance of new molecular species (presumably cancer-derived) in the same cancer-bearing animals. While these initial studies have been carried out in animals, the results are considered to be indicative of results which would be obtained using samples obtained from human patients.

### EXAMPLE 1

### Detection of Immune System Associated Molecules in Serum

Previous serological studies have shown that total T-cell derived glycolipids are enhanced about 100-fold in the serum of dextran sulfate treated mice (Parish et al, Cell. Immunol 33, 134-144, 1977). Conversely, such glycolipids are lacking from the sera of T-cell deficient nude mice (Parish et al, Immunogenetics 3, 129-137, 1976).

Parish et al also reported that immune system derived glycolipids can be extracted from serum by an ammonium sulfate/pyridine method (Parish et al, Immunogenetics 3, 455-463, 1976).

Accordingly, we extracted serum samples from normal mice, nude mice, and normal mice that had been injected with dextran sulfate, using ammonium sulfate/pyridine, and analyzed those samples by MALDI-TOF MS, to determine whether or not mass spectrometry was applicable to the analysis of glycolipid and oligosaccharides in sera, and whether or not the process could be applied to the identification of cancer markers.

Materials and Methods

1. Serum Samples

Blood was collected from normal BALB/c mice or BALB/c mice injected ip 4 days previously with 1 mg of 500 kDa dextran sulfate. Blood was also collected from T-cell deficient Swiss nude mice. Following collection blood was incubated at 37° C. for 30 min, stored at 4° C. overnight, and the serum collected following centrifugation.

2. Fractionation of Serum—Ammonium Sulfate/Pyridine Method

Serum proteins were precipitated using saturated ammonium sulfate and the supernatant subsequently desalted using pyridine, as previously published (Parish et al, Immunogenetics 3, 455-463, 1976). The pyridine was removed by evaporation and the residue resuspended in chloroform/methanollwater [2/43/55 (v/v/v)]. The suspension was filtered through a 0.2 μm filter and the filtrate was applied twice onto a pre-equilibrated C₁₈ Seppak cartridge (Waters, Taunton, Mass.). The eluate (non-adsorbed fraction or flow through) was collected and analyzed using MALDI-TOF MS as described below. The cartridge was then sequentially eluted with 2 ml methanol/water solution, followed by 2 ml methanol, followed by 2 ml chloroform/methanol, followed by 2 ml chloroform. All fractions were collected separately and analyzed using MALDI-TOF MS as described below.

3. MALDI-TOF MS Analysis

To prepare samples for mass spectrometry, the fractions were dried in vacuo. The flow through fraction and the methanovwater fraction were dissolved in water (200 μl), dialyzed extensively against water using a 1 kDa molecular weight cut off dialysis membrane, and dried by evaporation. All fractions were re-dissolved in 10 μl of the relevant solvent for loading into the mass spectrometer.

Fractions prepared as described supra (1 μl) and mixed, by vortex, with matrix solution [1 μl of a 3.5 mg/ml solution of 2-(4-hydroxyphenylazo) benzoic acid (HABA) in methanol]. The mixture (1 μl) was loaded onto a sample plate having 96 loading positions, and dried at room temperature. The sample plate was then loaded into the MALDI-TOF MS (TofSpec-2e; Micromass, Manchester, UK). A nitrogen laser (337 nm) was used for ionization, and the analysis was carried out in the linear negative ion mode. Data are presented as molecular mass profiles in Da, with peak heights being depicted as the percentage height of the most abundant molecular species detected in the sample.

Results

Initial studies examined whether MALDI-TOF MS could be used to detect molecular species that are immune system derived and/or require activated T-cell function for their expression.

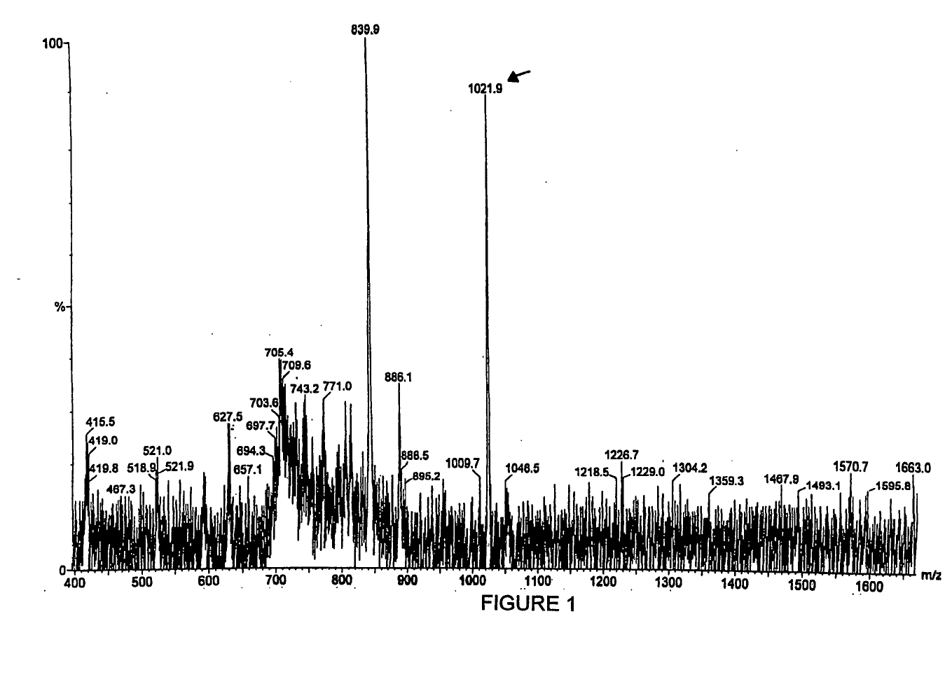

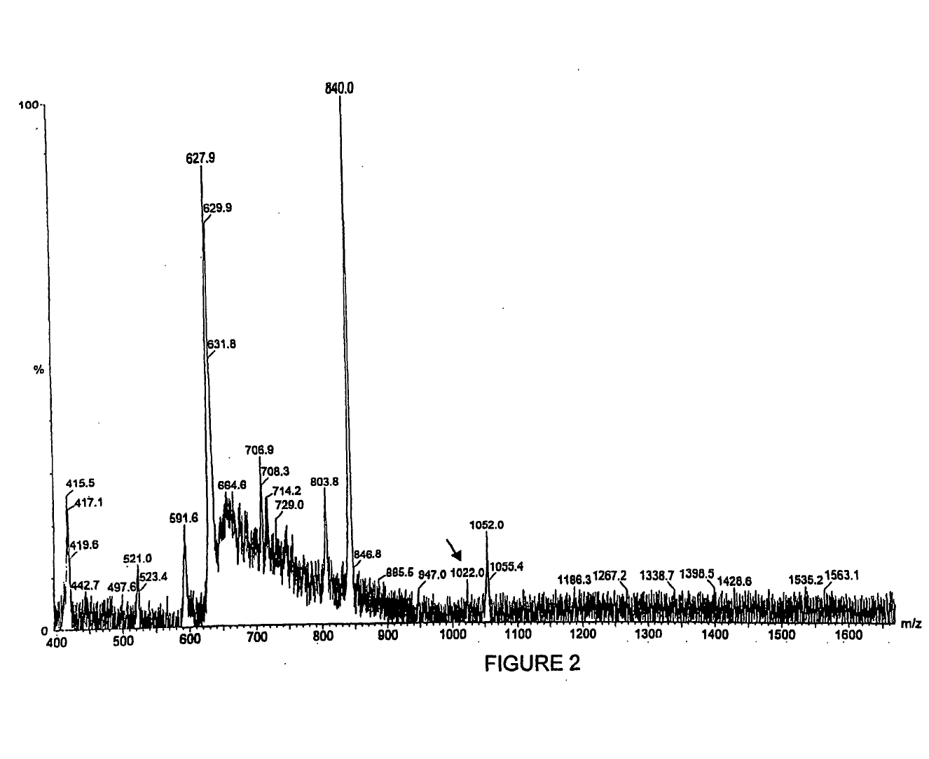

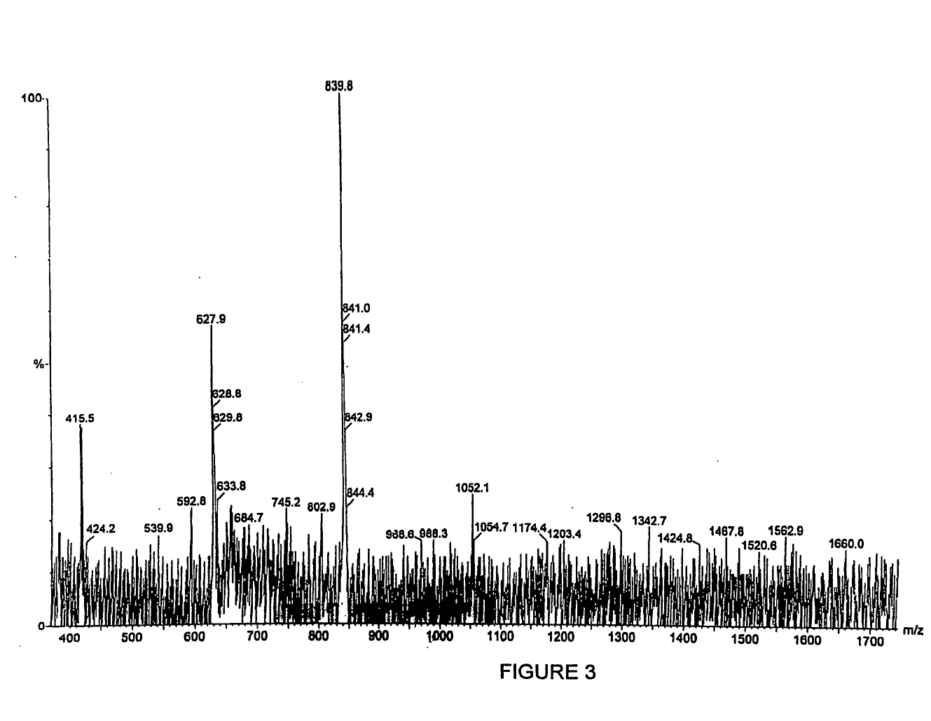

We found that the flow through fraction (i.e. the fraction that did not adsorb to the C₁₈ Seppak column) from the sera of dextran sulfate-treated BALB/c mice contained a very prominent species having a molecular mass of about 1022 Da, when analyzed by MALDI-TOF MS (FIG. 1). In contrast, this molecular mass species, although detectable, was present at much lower levels in the flow through fraction derived from untreated BALB/c mice (FIG. 2), and was not detectable at all by MALDI-TOF MS in the serum of nude mice (FIG. 3).

The data presented in FIGS. 1 to 3 indicate that the ˜1022 Dalton species is immune system dependent. However, the fact that this species did not bind to the C₁₈ Seppak cartridge under the conditions described herein suggests that it is a serum oligosaccharide.

### EXAMPLE 2

### Cancer Markers that are Reduced in the Serum of Animals Having Tumors

Materials and Methods

1. Serum Samples

Blood was collected from female Fisher 344 rats carrying the highly metastatic rat mammary adenocarcinoma 13762 MAT (Parish et al, Int. J. Cancer 40, 511-518, 1987). To induce tumors in animals, tumor cells were maintained in vitro as previously described (Parish et al, Int. J. Cancer 40, 511-518, 1987), and rats (10-13 weeks of age) were injected i.v. with 2×10⁵ 13762 MAT cells in 0.6 ml RPMI 1640 medium containing 10% (v/v) FCS. Blood was collected 13 days following tumor cell injection. At this stage a number of small secondary tumors are seen in the lungs of the rats, but they show no signs of distress from the tumor. Sera were prepared from the blood as described in Example 1.

2. Fractionation of Serum

Sera from tumor bearing rats were fractionated by the ammonium sulfate/pyridine method as described in Example 1.

3. MALDI-TOF MS Analysis

Mass spectrometry, including sample preparation and loading and data analyses, were performed as described in Example 1.

Results

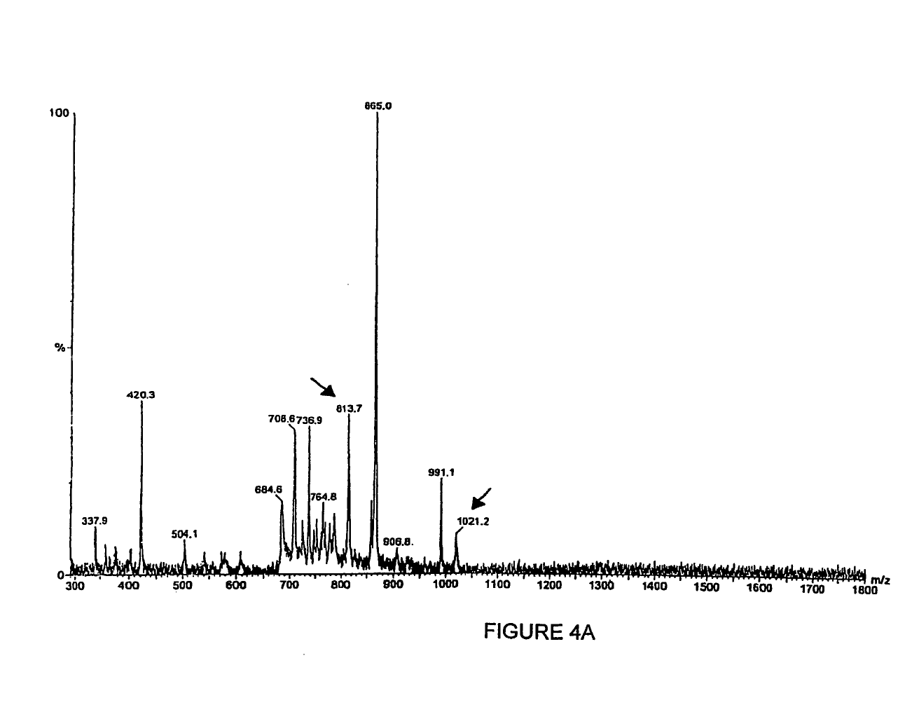

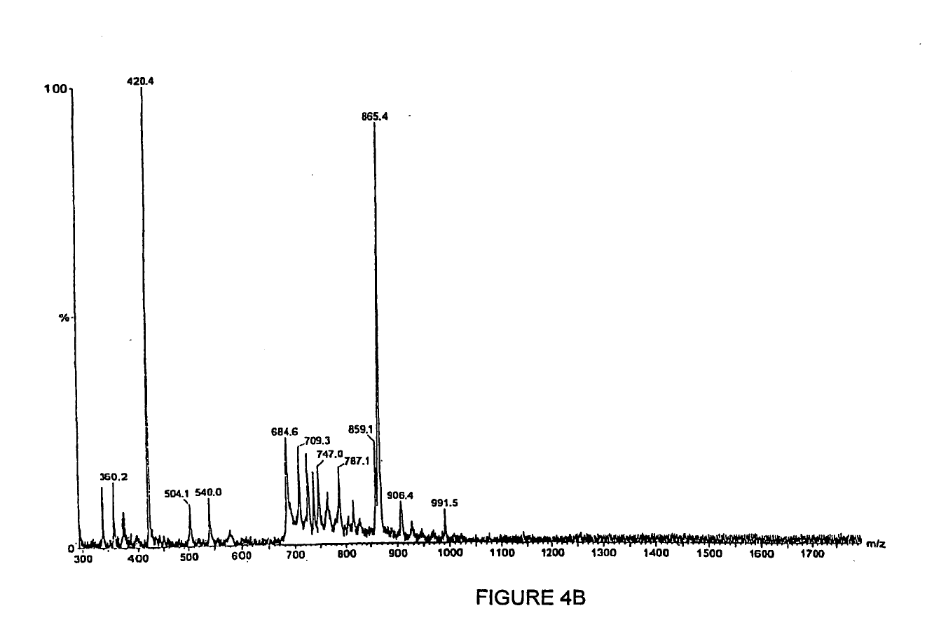

Data presented in FIG. 4A and FIG. 4B show the mass spectrometry profiles of C₁₈ Seppak flow through fractions of sera from normal and tumor-bearing rats. Two molecular species, having molecular masses of about 814 Da and 1021 Da, are reduced in the sera of tumor-bearing animals compared to normal animals (compare FIGS. 4A and 4B). The fact that these species do not bind to the C₁₈ Seppak cartridge under the conditions described herein suggests that they are serum oligosaccharides. The 1021 Dalton species is the same as the 1022 Dalton species identified as being immune system associated in Example 1 supra.

### EXAMPLE 3

### Cancer Markers that are Enhanced in the Serum of Animals Having Tumors

Materials and Methods

1. Serum Samples

Blood was collected from normal and tumor bearing rats as described in Example 2.

2. Fractionation of Serum—Chloroform:Methanol Method

Serum (1 ml) from tumor bearing animals was dried in vacuo. Chloroform:methanol solution (2 ml) was added to each serum sample, and the mixtures incubated overnight at 4° C. with vigorous stirring, to extract glycolipid. The mixtures were centrifuged and the chloroform/methanol phases were collected. The extractions were repeated 4 more times, each extraction being for 2 hr at 4° C. The chloroform/methanol phases for each extract were pooled, and dried in vacuo. Residues were resuspended in chloroform/methanol/water solution [2/43/55 (v/v/v)]. The suspensions were filtered through a 0.2 μm filter and the filtrates were applied twice onto pre-equilibrated C₁₈ Seppak cartridges. The eluates (non-adsorbed fractions or flow through fractions) were collected. Each cartridge was then sequentially eluted with 2 ml methanol/water solution, followed by 2 ml methanol, followed by 2 ml chloroform/methanol, followed by 2 ml chloroform. All fractions were collected separately.

3. MALDI-TOF MS Analysis

Mass spectrometry, including sample preparation and loading and data analyses, were performed as described in Example 1.

Results

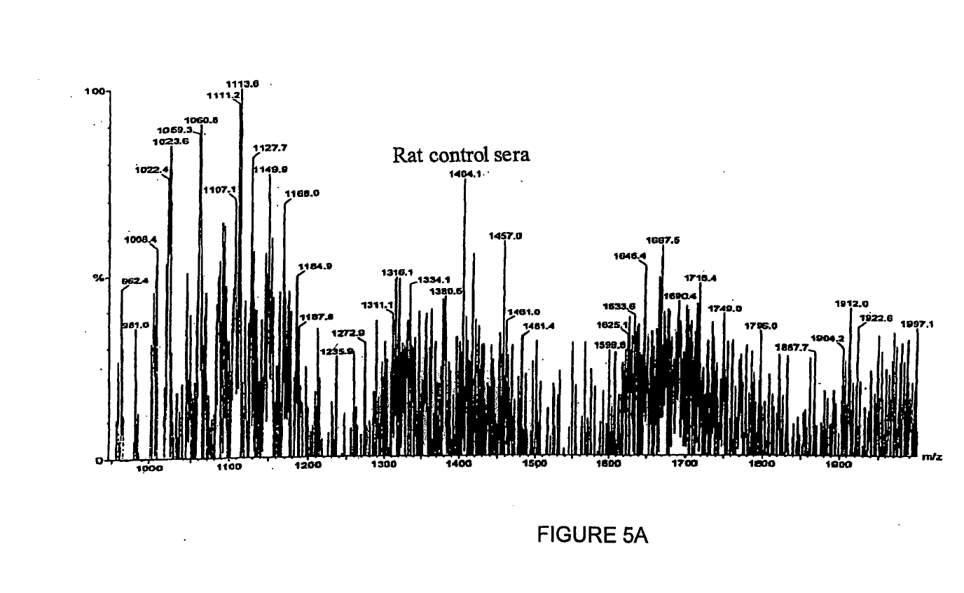

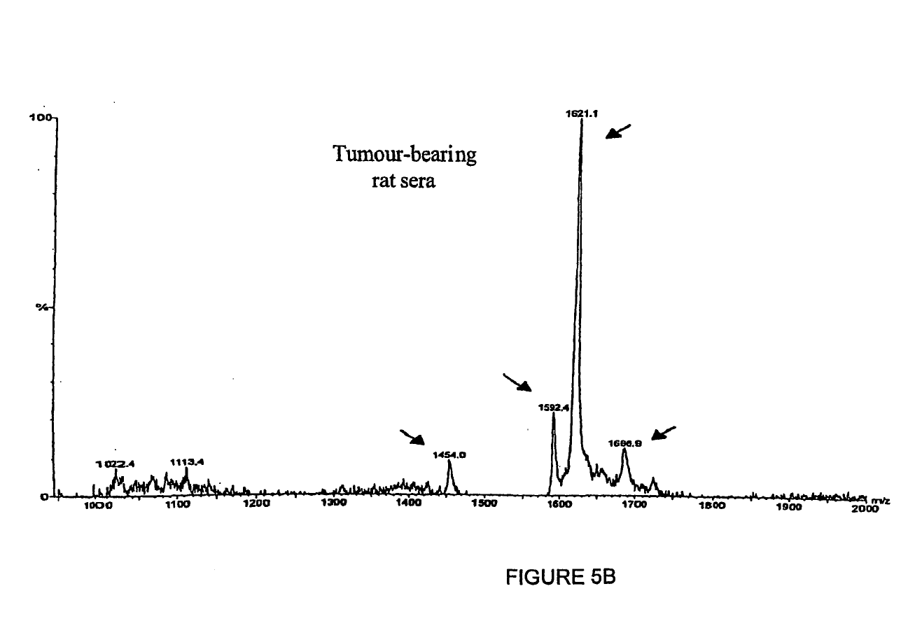

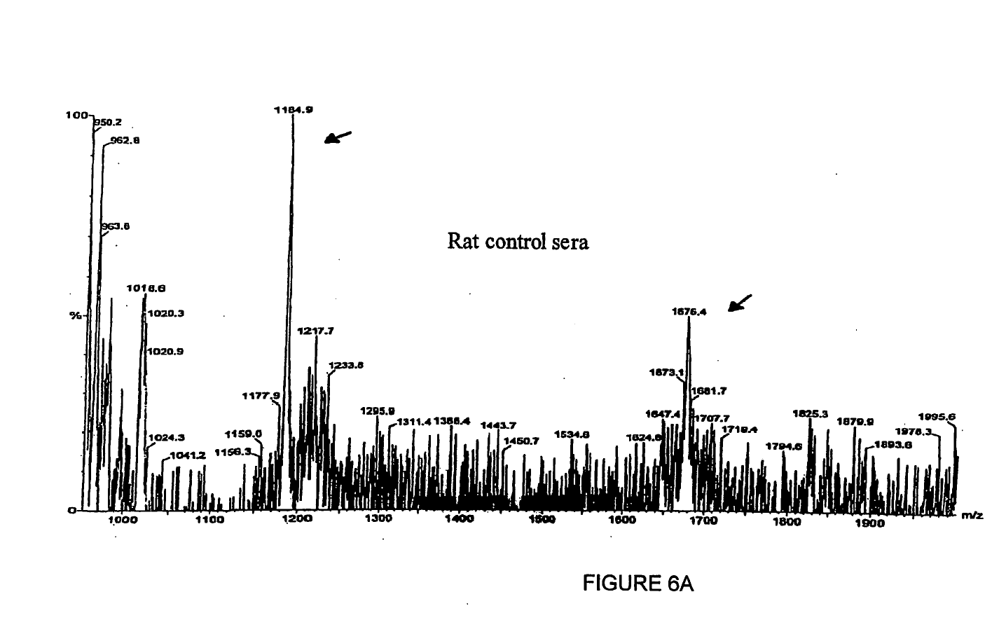

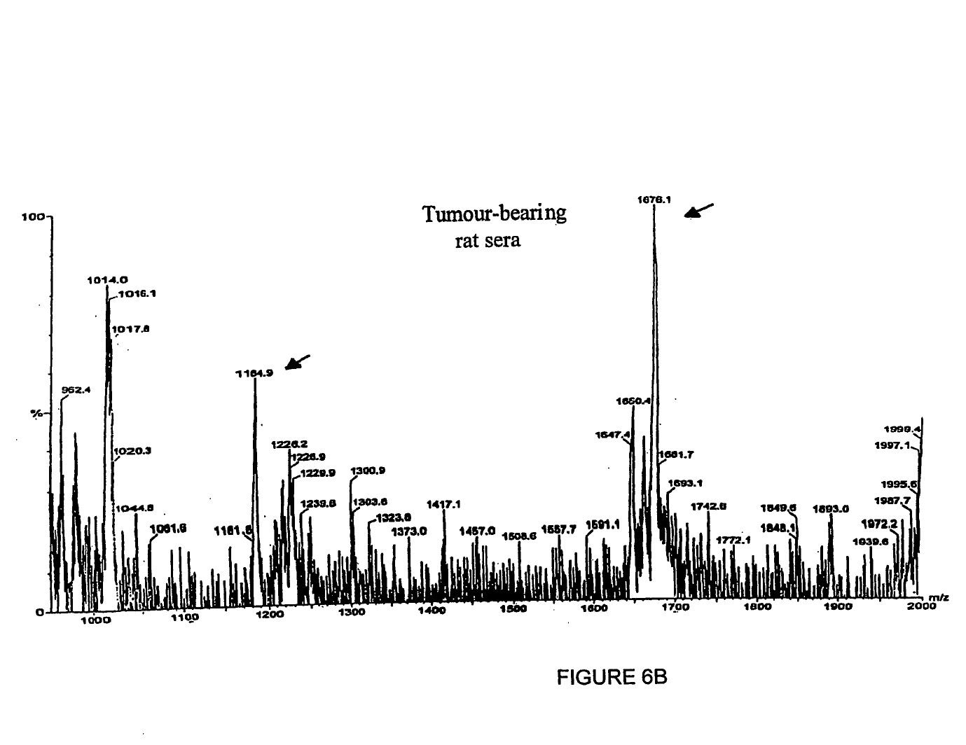

Tumor-associated molecular species were identified in the chloroform/methanol extracts of serum fractions (FIG. 5B) and in the methanol extracts of serum fractions (FIG. 6B), from tumor-bearing rats. As these fractions contained molecular species that bound to the C₁₈ Seppak cartridges, they are glycolipids, preferably gangliosides. These species were also present at very much reduced levels, or non-detectable by MALDI-TOF MS in the sera of healthy rats that did not carry tumors (FIGS. 5A and 6A).

More specifically, the chloroform/methanol eluates of tumor-bearing animals contained four prominent species having molecular masses as determined by MALDI-TOF MS of about 1454 Da, 1592 Da, 1621 Da, and 1686 Da, respectively, that were not detectable in control sera samples (FIG. 5A). In fact, a large number of background peaks were observed in the spectrum derived from chloroform/methanol eluates of normal rats, with no predominant species detected (FIG. 5A). These data demonstrate unequivocally that cancer markers can be detected in the serum of cancer bearing animals by MALDI-TOF MS.

The methanol eluate of tumor-bearing animals contained a predominant glycolipid/ganglioside having a molecular mass as determined by MALDI-TOF MS of about 1676 Da (FIG. 6B). This species was also present in the serum of control rats (FIG. 6A), albeit at a much lower level than in the tumor-derived samples. The modified level of the 1676 Da species is particularly evident when the peak height of this species is compared to the peak height of the 1185 Da glycolipid in both spectra (FIGS. 6A and 6B). Accordingly, the 1676 Da species is enhanced in the sera of tumor-bearing animals.

It is possible that the 1676 Da glycolipid is normally secreted by proliferating cells and, therefore, the presence of proliferating cancer cells in an animal results in an increase in the serum levels of this molecule.

We claim:

1. A method of identifying a cancer marker comprising:
(i) separating a blood fraction from a human or animal subject having a cancer by mass spectrometry;
(ii) separating a blood fraction from a healthy human or animal subject by mass spectrometry; and
(iii) comparing the profile of molecular species at (i) and (ii) and identifying those molecular species having a modified level at (i) compared to (ii), wherein an enhanced or reduced level of said molecular species indicates that the molecular species is a cancer marker.

2. The method of claim 1 wherein the mass spectrometry comprises Matrix Assisted Laser Desorption/Ionisation Time of Flight Mass Spectrometry (MALDI-TOF MS).

3. The method of claim 1 wherein the mass spectrometry comprises electrospray mass spectrometry.

4. The method of claim 1 wherein the cancer marker is a glycolipid.

5. The method of claim 4 wherein the glycolipid is a ganglioside.

6. The method of claim 1 wherein the cancer marker is an oligosaccharide.

7. The method of claim 1 wherein the cancer is selected from the group consisting of ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans.

8. The method of claim 1 wherein the cancer is selected from the group consisting of lymphoma (several), melanoma, sarcoma, and carcinoma of non-human animals.

9. The method of claim 7 wherein the carcinoma is adenocarcinoma.

10. The method of claim 1 wherein the blood fraction is a serum fraction.

11. The method of claim 1 further comprising the first step of obtaining the blood fraction from the subject having cancer.

12. The method of claim 1 further comprising the first step of obtaining the blood fraction from the healthy subject.

13. The method of claim 1 further comprising determining the abundance of the cancer marker in the blood fraction from the subject having cancer or the blood fraction from the healthy subject or determining the relative abundance of a molecular species in said blood fractions.

14. A method for identifying a cancer marker that is indicative of a specific cancer, said method comprising:
(i) separating a blood fraction from a human or animal subject having a cancer by mass spectrometry;
(ii) separating a blood fraction from a human or animal subject having a cancer other than the cancer at (i) by mass spectrometry;
(iii) separating a blood fraction from a healthy human or animal subject by mass spectrometry; and
(iv) comparing the profile of molecular species at (i) and (ii) and (iii) and identifying those molecular species having a modified level at (i) or (ii) when compared to (iii), wherein said modified level indicates that the molecular species is a cancer marker that is indicative of a specific cancer.

15. The method of claim 14 wherein the mass spectrometry comprises Matrix Assisted Laser Desorption/Ionisation Time of Flight Mass Spectrometry (MALDI-TOF MS).

16. The method of claim 14 wherein the mass spectrometry comprises electrospray mass spectrometry.

17. The method of claim 14 wherein the cancer marker is a glycolipid.

18. The method of claim 17 wherein the glycolipid is a ganglioside.

19. The method of claim 14 wherein the cancer marker is an oligosaccharide.

20. The method of claim 14 wherein the cancer marker is indicative of a specific cancer selected from the group consisting of ovarian cancer, colon cancer, breast cancer, pancreatc-cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans.

21. The method of claim 14 wherein the cancer marker is indicative of a specific cancer selected from the group consisting of lymphoma (several), melanoma, sarcoma, and carcinoma of non-human animals.

22. The method of claim 21 wherein the carcinoma is adenocarcinoma.

23. The method of claim 14 wherein the blood fraction is a serum fraction.

24. The method of claim 14 further comprising the first step of obtaining a blood fraction from any one or more of said subjects.

25. The method of claim 14 further comprising determining the abundance or relative abundance of a cancer marker in any one or more of said blood fractions.

26. A method for identifying a cancer marker that is indicative of a specific cancer, said method comprising:
(i) separating by mass spectrometry a panel of blood fractions from a human or animal subject wherein each member of said panel is from a subject having a distinct cancer;
(ii) separating a blood fraction from a healthy human or animal subject by mass spectrometry;
(iii) comparing the profiles of molecular species from each member of said panel of blood fractions at (i) to each other and to the profile of molecular species from the blood fraction at (ii); and
(iv) identifying from (iii) those molecular species having a modified level in one member of said panel at (i) when compared to the profile of the blood fraction at (ii), wherein said modified level indicates that the molecular species is a cancer marker that is indicative of a specific cancer.

27. The method of claim 26 wherein the mass spectrometry comprises Matrix Assisted Laser Desorption/Ionisation Time of Flight Mass Spectrometry (MALDI-TOF MS).

28. The method of claim 26 wherein the mass spectrometry comprises electrospray mass spectrometry.

29. The method of claim 26 wherein the cancer marker is a glycolipid.

30. The method of claim 29 wherein the glycolipid is a ganglioside.

31. The method of claim 26 wherein the cancer marker is an oligosaccharide.

32. The method of claim 26 wherein the cancer marker is indicative of a specific cancer selected from the group consisting of ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans.

33. The method of claim 26 wherein the cancer marker is indicative of a specific cancer selected from the group consisting of lymphoma (several), melanoma, sarcoma, and carcinoma of non-human animals.

34. The method of claim 33 wherein the carcinoma is adenocarcinoma.

35. The method of claim 26 wherein the blood fraction is a serum fraction.

36. The method of claim 26 further comprising the first step of obtaining a blood fraction from any one or more subjects.

37. The method of claim 26 further comprising determining the abundance or relative abundance of a cancer marker in any one or more of said blood fractions.

38. A method for diagnosing or detecting cancer in a human or animal subject comprising:
(i) separating a test sample comprising a blood fraction from a human or animal subject suspected of having a cancer by mass spectrometry;
(ii) separating a control sample comprising a blood fraction from a healthy subject by mass spectrometry; and
(iii) comparing the level of a cancer marker at (i) and (ii) wherein an enhanced or reduced level of said cancer marker in the test sample compared to the control sample indicates that the subject at (i) has a cancer.

39. The method of claim 38 wherein the mass spectrometry comprises Matrix Assisted Laser Desorption/Ionisation Time of Flight Mass Spectrometry (MALDI-TOF MS).

40. The method of claim 38 wherein the mass spectrometry comprises electrospray mass spectrometry.

41. The method of claim 38 wherein the cancer marker is a glycolipid.

42. The method of claim 41 wherein the glycolipid is a ganglioside.

43. The method according to claim 41 or 42 wherein the glycolipid has a molecular mass as determined by mass spectrometry selected from the group consisting of:
(i) a molecular mass in the range of 1439 to 1459 Da (average mass 1454 Da);
(ii) a molecular mass in the range of 1587 to 1597 Da (average mass 1592 Da);
(iii) a molecular mass in the range of 1616 to 1626 Da (average mass 1621 Da);
(iv) a molecular mass in the range of 1671 to 1681 Da (average mass 1676 Da); and
(v) a molecular mass in the range of 1681 to 1691 Da (average mass 1686 Da).

44. The method of claim 38 wherein the cancer marker is an oligosaccharide.

45. The method according to claim 44 wherein the oligosaccharide has a molecular mass as determined by mass spectrometry selected from the group consisting of:
(i) a molecular mass in the range of 809 to 819 Da (average mass 814 Da); and
(ii) a molecular mass in the range of 1016 to 1026 Da (average mass 1021 Da).

46. The method of claim 38 wherein the subject suspected of having a cancer and the healthy subject are human.

47. The method of claim 46 wherein the cancer is selected from the group consisting of ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma.

48. The method of claim 38 wherein the subject suspected of having a cancer and the healthy subject are non-human animals.

49. The method of claim 48 wherein the cancer is selected from the group consisting of lymphoma (several), melanoma, sarcoma, and carcinoma.

50. The method of claim 49 wherein the carcinoma is adenocarcinoma.

51. The method of claim 38 wherein the blood fraction is a serum fraction.

52. The method of claim 38 further comprising the first step of obtaining a blood fraction from any one or more subjects.

53. The method of claim 38 further comprising determining the abundance or relative abundance of a cancer marker in any one or more of said blood fractions.

54. The method of claim 38 when used to monitor the progress of a cancer in a human or animal subject.

55. A method for diagnosing or detecting cancer in a human or animal subject comprising:
(i) performing the method of claim 1 or 14 to identify a cancer marker; and
(ii) determining the level of said cancer marker in a blood fraction from a human or animal subject suspected of having a cancer, wherein a modified level of said cancer marker compared to a healthy blood fraction indicates that the subject has cancer.

56. The method of claim 55 wherein the level of the cancer marker in a blood fraction is determined by a process selected from the group consisting of: mass spectrometry, hydrophobic interaction chromatography, size exclusion chromatography, ion exchange chromatography, and immunoassay.

57. An isolated cancer marker selected from the group consisting of:
(i) a glycolipid having a molecular mass in the range of 1439 to 1459 Da (average mass 1454 Da);
(ii) a glycolipid having a molecular mass in the range of 1587 to 1597 Da (average mass 1592 Da);
(iii) a glycolipid having a molecular mass in the range of 1616 to 1626 Da (average mass 1621 Da);
(iv) a glycolipid having a molecular mass in the range of 1671 to 1681 Da (average mass 1676 Da);
(v) a glycolipid having a molecular mass in the range of 1681 to 1691 Da (average mass 1686 Da);
(vi) an oligosaccharide having a molecular mass in the range of 809 to 819 Da (average mass 814 Da); and
(vii) an oligosaccharide having a molecular mass in the range of 1016 to 1026 Da (average mass 1021 Da).

58. The method of claim 57 wherein the cancer marker is a marker for a carcinoma.

59. A method of identifying a cancer marker for a carcinoma comprising:
(iv) separating a blood fraction from a subject having a carcinoma by Matrix Assisted Laser Desorption/lonisation Time of Flight Mass Spectrometry (MALDI-TOF MS);
(v) separating a blood fraction from a healthy subject by MALDI-TOF MS; and
(vi) comparing the profile of molecular species at (i) and (ii) and identifying those molecular species having a modified level at (i) compared to (ii), wherein an enhanced or reduced level of said molecular species indicates that the molecular species is a cancer marker for carcinoma.

60. A method for diagnosing or detecting a carcinoma in subject comprising:
(i) separating a test sample comprising a blood fraction from a subject suspected of having a cancer by Matrix Assisted Laser Desorption/Ionisation Time of Flight Mass Spectrometry (MALDI-TOF MS);
(ii) separating a control sample comprising a blood fraction from a healthy subject by MALDI-TOF MS; and
(iii) comparing the level of a cancer marker at (i) and (ii) wherein an enhanced or reduced level of said cancer marker in the test sample compared to the control sample indicates that the subject at (i) has a cancer and wherein the cancer marker is selected from the group consisting of:
(a) a glycolipid having a molecular mass in the range of 1439 to 1459 Da (average mass 1454 Da);
(b) a glycolipid having a molecular mass in the range of 1587 to 1597 Da (average mass 1592 Da);
(c) a glycolipid having a molecular mass in the range of 1616 to 1626 Da (average mass 1621 Da);
(d) a glycolipid having a molecular mass in the range of 1671 to 1681 Da (average mass 1676 Da);
(e) a glycolipid having a molecular mass in the range of 1681 to 1691 Da (average mass 1686 Da);
(f) an oligosaccharide having a molecular mass in the range of 809 to 819 Da (average mass 814 Da); and
(g) an oligosaccharide having a molecular mass in the range of 1016 to 1026 Da (average mass 1021 Da).
